(19) United States
(12) Patent Application Publication
(10) Pub. No.: US 2022/0047659 A1
(43) Pub. Date: Feb. 17, 2022

(54) SYMBIOTIC SUPPLEMENT FORMULATION FOR IMPROVING INTESTINAL MICROBIOTA

(71) Applicant: CENTRO DE RETINA MEDICA Y QUIRURGICA, S.C., Zapopan (MX)

(72) Inventors: Arturo SANTOS GARCIA, Zapopan (MX); Renata Yoshie SAMPAIO SANODA, Zapopan (MX); Maria Jose RIVAS ARREOLA, Zapopan (MX)

(21) Appl. No.: 17/415,723

(22) PCT Filed: Nov. 22, 2019
(86) PCT No.: PCT/MX2019/000127

§ 371 Date: Jun. 18, 2021

(30) Foreign Application Priority Data
Dec. 18, 2018 (MX) MX/A/2018/015947

(51) Int. Cl. A61K 36/185; A61K 31/733; A61K 35/747; A61K 35/745; A61K 31/4172; A61K 31/198; A61K 33/00; A61K 31/4415; A61K 31/714; A61K 31/525; A61K 31/455; A61K 31/519; A61K 31/07; A61K 31/197; A61K 31/51; A61K 31/355; A61K 31/122; A61K 31/593; A61K 31/375; A61K 31/4188; A61K 47/36; A23L 33/105; A23L 33/135; A23L 33/175; A23L 33/16; A23L 33/155; A23L 33/22

(52) U.S. Cl. CPC A61K 36/185; A23Y 2300/55; A61K 35/747; A61K 35/745; A61K 31/4172; A61K 31/198; A61K 33/00; A61K 31/4415; A61K 31/714; A61K 31/525; A61K 31/455; A61K 31/519; A61K 31/07; A61K 31/197; A61K 31/51; A61K 31/355; A61K 31/122; A61K 31/593; A61K 31/375; A61K 31/4188; A61K 47/36; A23L 33/105; A23L 33/135; A23L 33/175; A23L 33/16; A23L 33/155; A23L 33/22; A23V 2002/00; A23Y 2220/17; A23Y 2220/73; A23Y 2220/49; A23Y 2220/67; A61K 31/733

(57) ABSTRACT

Described is a symbiotic formulation for the oral consumption of probiotics and prebiotics with an aqueous extract of roselle, with vitamins and proteins for humans. The invention belongs to the field of nutrition and has been developed to help to maintain a healthy digestive system by restoring and/or improving the intestinal microbiota. The purpose of the antioxidants and the synergic effect of the symbiotic formulation is to reduce the inflammatory process so as to facilitate the absorption of nutrients and thus improve the good nutritional state of people. An oral symbiotic formulation based on an aqueous roselle extract with encapsulated probiotics and which can contribute to the consumption of antioxidants of natural origin, the ingestion of proteins and micronutrients for re-establishing or improving the state of the intestinal microbiota, as well as the nutritional state of adults and older people, has not been found in the prior art.

### FIELD OF THE INVENTION

The present invention relates to the field of medicine and nutrition, specifically to a formulation characterized in that it is a source of probiotics, prebiotics, vitamins and proteins with antioxidants from an aqueous extract of roselle to assist in the preservation of intestinal microbiota, strengthening of the immune system and tissue repair.

### BACKGROUND OF THE INVENTION

A good nutritional state involves meeting requirements for essential nutrients and having a strong immune system to protect the organism from developing chronic diseases, such diseases being in some cases related to diet and lifestyle. The current prevalence of chronic degenerative diseases requires a change in the diet of the population in order to adequately meet the required levels of energy, strengthen the immune system and improve nutrient absorption.

Health measures such as urban sanitation, clean drinking water, sewer systems, vaccines, combating infections with antibiotics and recognizing bacteria as a cause of disease have had a significant impact on the history of mankind.

However, since the beginning of human existence, living beings have been in close contact with diverse bacterial communities. The stability of the association between the different bacterial species and human beings suggests the existence of an adaptation that benefits both. Although medicine has benefited from recognizing and combating the harmful microbes that cause infectious diseases, scientific advances demonstrate that the vast majority of microorganisms living alongside man help humans achieve good physiological development.

The community of living microorganisms hosted in the human digestive tract and that has adapted to live on the surface of the intestine for millennia is known as intestinal microbiota. The intestine of an individual begins life sterile and acquires microbial colonization immediately after birth through the mother and the early environment. The vast majority of the organisms of the microbiota are always present in the intestine, whereas a portion known as “transiting microorganisms” live there temporarily, being basically ingested through food and drink. These collected microbes are closely interdependent and form the intestinal microbiota. From being considered an accompanying commensal organism said microbiota is now considered a “metabolic organ,” with functions in nutrition, immune regulation and systemic inflammation.

The human intestine is seen as an ecosystem because of the number of microorganisms that live there, estimated at between 10 trillion and 100 trillion microorganisms. These microorganisms are of different bacterial species responsible for regulating the environment of the host to facilitate efficient nutrient absorption and promote health, being involved in the regulation of metabolic disease mechanisms, the immune system and neurological activities.

It has been found that healthy people maintain high levels and a great variety of microorganisms in the intestine whereas people with a disease such as obesity or a chronic inflammatory process have fewer species of microorganisms. These microorganisms interact dynamically as said microorganisms are responsible for the biotransformation of the components of foods, facilitating digestive processes and absorption. These actions help modulate the immune system of the host and the metabolic responses.

It has also been identified that diet modulates the composition and functionality of the intestinal microbiota, establishing a system of mutual interaction and interdependence. Macronutrients, fibers, as well as polyphenols and prebiotics are important and powerful components that give form to the composition of the intestinal microbiota.

In recent years, research in this field has shown that the function of the intestine depends not only on said intestine as a mucosal barrier and secretion barrier through its glands, but that the intestinal microbiota also acts as an additional organ. Experiments with animals reared in a sterile environment are a striking example. These animals have a lower body weight and a lower vital organ weight than animals reared in a normal environment. Animals reared in a sterile environment ingest a larger amount of food and grow far less. This clearly indicates that bacteria are very important for nutrition and body growth. Another piece of information from these studies indicates that animals reared in a sterile environment have atrophic lymph nodes and fewer cells in the immune system, showing that the bacteria of the intestinal microbiota are necessary for the development of the immune system of the individual. Among the most important probiotics for human health are:

| Probiotic genus | Species involved |
|---|---|
| Lactobacillus | L. plantarum, L. paracasei, L. acidophilus, |
| | L. casei, L rhamnosus, L. crispatus, |
| | L. gasseri, L. reuteri, L. bulgaricus |
| Propionibacterium | P. jensenii, P. freudenreichii |
| Peptostreptococcus | P. productus |
| Bacillus | B. coagulans, B. subtilis, B. laterosporus |
| Lactococcus | L. lactis, L. reuteri, L. rhamnosus, L. casei, |
| | L. acidophilus, L. curvatus, L. plantarum |
| Enterococcus | E. faecium |
| Pediococcus | P. acidilactici, P. pentosaceus |
| Streptococcus | S. sanguis, S. oralis, S. mitis, |
| | S. thermophilus, S. salivarius |
| Bifidobacterium | B. longum, B. catenulatum, B. breve, |
| | B. animalis, B. bifidum |
| Bacteroides | B. uniformis |
| Akkermansia | A. muciniphila |
| Saccharomyces | S. boulardii |

Lactobacilli and Bifidobacteria are natural components of the gut microbiota in humans. An example of the health effects linked to a good state of intestinal microbiota is that the presence of Lactobacillus casei has been shown to reduce the secretion of TNF-α in the inflamed ileum of patients with Crohn's disease. It has also been shown that Lactobacilli and Bifidobacteria regulate fat storage, reduce cholesterol and promote intestinal angiogenesis. It has been suggested that the beneficial effect of probiotics may be due, at least in part, to interference with the innate immune system and possibly the orientation of adaptive immunity.

Such is the beneficial effect of using probiotics that said effect has been assessed during pregnancy and it has been demonstrated that probiotics have the potential to influence the fetal immune parameters, as well as immunomodulatory factors in milk.

Prebiotics are natural compounds of some plant foods or alternatively are industrially processed substances. Said compounds are non-digestible and are used by the microorganisms that inhabit the digestive systems of people as a substrate and are recognized as health-promoting food ingredients, given that the consumption thereof is associated with the production of short-chain fatty acids and polyamines through the fermentation of carbohydrates; this improves intestinal function and mobility as it stimulates and promotes the growth of microorganisms and the activity thereof.

Agave inulin is a prebiotic which consists of an inulin-levan fructose polymer; its structures are linked by ρ-2,1 and β-2,6 bonds with branches and terminal glucoses hydrolyzed by inulinase and levanase enzymes of the probiotic bacteria. Although a wide variety of polymeric compounds are now present on the market as prebiotics, agave inulin is one of the few that demonstrates potential on Bifidobacterium species, specifically on B. longum. The proposed formulation that is the subject of this document includes Bifibobacteria Longum as this is the microorganism that mostly inhabits the human large intestine and its presence is essential in order to eliminate pathogenic bacteria as said microorganism reduces fecal ammonia. The dynamic synergy between prebiotics and probiotics is the source of the concept of symbiotic foods.

It has been demonstrated that extracts of roselle, Hibiscus sabdariffa, have antioxidant compounds such as phenols and flavonoids (antioxidants), vitamins and minerals. Such extracts are used traditionally in foods, cold and hot drinks, as a flavoring and in traditional medicine. The beneficial functions reported for such extracts include antibacterial, antioxidant and/or diuretic effects, a lipid metabolism support effect and a vasodilatory effect.

In extensive and varied studies of the effects of probiotics, supplements with prebiotics or symbiotics on oxidative stress parameters, it was found that these types of supplements improve the effect and bioavailability of antioxidant enzymes and compounds. These analyses show that incorporating supplements with probiotics or symbiotics in the diet may improve the resistance of the body to oxidizing agents. Inflammation is a physiological response of the body when internal injury or threats from external factors are detected. The antioxidants present in extracts of roselle, Hibiscus sabdariffa, have been shown to reduce levels of tumor necrosis factor α and also to modulate cytokinins, which represents a significant anti-inflammatory effect. Patients with diseases who suffer from intestinal inflammation have a greater risk of malnutrition as nutrient absorption is not optimal; these patients therefore require nutritional support.

Micronutrients are essential in the diet and have a fundamental role in disease prevention. The chemical reactions of the metabolic pathways cannot take place without an adequate level of micronutrients being present.

Some sectors of the population do not have an adequate intake of micronutrients, either for economic reasons or due to an unsuitable balance of food choices. Within the micronutrient group, vitamins (water-soluble or fat-soluble) can be identified. Vitamins and their metabolites act as coenzymes or as active compounds within biochemical reactions. Riboflavin and niacin act as a coenzyme in the chain. Folic acid forms part of methyl group transfer. These reactions are essential for intermediary metabolism, ensuring the utilization of the major nutrients and supporting the production of energy, proteins and nucleic acids.

Proteins are macronutrients that must be included in the diet as they are responsible for muscle maintenance and regulating hormone and neurotransmitter segregation. Proteins make up the macromolecular structures of organelles, mediate enzyme activity, drive signaling cascades, drive transcription and translation, form transport mechanisms and are fundamental components of most (if not all) the basic operations of life. Most proteins require a complex folding process after being synthesized, a process which distinguishes said proteins from other classes of biomolecules in terms of the complexity involved. Managing the folding process (proteostasis) requires wide-ranging guidance of a quality control network comprising about 800 proteins in humans.

Some people in good health but with factors for developing chronic degenerative diseases have some degree of undernourishment, muscular wasting or inflammation. To improve the quality of life of such people the intake of ketogenic amino acids, which are recognized for their great tissue repair power, is recommended.

The invention which has been developed and is described below, is a gelatinous formulation based on an aqueous extract of roselle, which has antioxidant and anti-inflammatory properties, contains agave inulin which acts as a prebiotic for the microorganisms contained therein. The probiotics in the formulation are encapsulated in a sodium alginate matrix to guarantee the viability and improve the bioavailability thereof; the symbiotic effect may help reduce the inflammatory response in conjunction with the antioxidants of the aqueous extract of roselle. These elements of the formulation, as well as the micronutrients and biomolecules, strengthen the immune system, improve and/or re-establish the intestinal microbiota improving nutrient absorption, and may therefore contribute to a better state of nutrition for people.

### OBJECT OF THE INVENTION

The object of the invention is to make available a symbiotic supplement, the formulation of which is administered orally to adult humans and the components of which are natural in origin.

The components of the formulation have antioxidant properties and essential elements for improving the nutritional state of people; in addition said formulation is based on an aqueous extract of roselle (Hibiscus sabdariffa) with prebiotics (agave inulin); the gelatin contains a mixture of probiotics (L. casei, L. rhamnosus, L. lactis, L. plantarum, B. longum) encapsulated in a polymer matrix (sodium alginate), with a mixture of micronutrients and macronutrients to enhance the nutritional state of people.

### DETAILED DESCRIPTION OF THE INVENTION

The details characteristic of the symbiotic supplement formulation for improving intestinal microbiota are set out clearly in the description that follows. The invention that has been developed consists of a symbiotic supplement in a gelatin formulation for oral administration to adults based on an aqueous extract of roselle (Hibiscus sabdariffa) of natural origin and contains a mixture of vitamins, proteins, probiotics and prebiotics.

The formulation has the potential to improve the nutritional state of people by providing a symbiotic effect that may help improve the diversity and quantity of intestinal microbiota; it may also facilitate the absorption of important compounds in the functioning and regulation of metabolism.

The aqueous extract of roselle has antioxidant properties as said extract has a high content of phenolic compounds. The antioxidant capacity of phenolic compounds is linked to mechanisms of action related to vasodilator, antithrombotic and anti-inflammatory properties. The traditional consumption of an infusion of H. sabdariffa is linked to various therapeutic properties such as the ability to absorb free radicals inhibiting, for example, LDL oxidation; to significantly improve blood pressure in pre-hypertensive and slightly hypertensive patients with type 2 diabetes.

The mixture of probiotics selected has the ability to restore the microbiota found naturally in adults. Said mixture of probiotics is encapsulated in a polymer matrix (sodium alginate) to improve the viability of the probiotics when passing through the digestive tract and in order that said probiotics are bioavailable in the small intestine. The aqueous extract of roselle gelatin contains high levels of prebiotics (agave inulin) to promote the growth of native microbiota and help ensure that the probiotics administered have significant viability levels. The purpose of this symbiotic effect is to restore the microbiota with a possible effect of enhancing the immune system, facilitating and improving nutrient absorption.

Until now, no substance that demonstrates undesirable side effects has been found and this formulation should therefore be used as suggested (once a day). It may also be used to maintain the intestinal microbiota.

Every 100 g of the formulation developed contains:
A soft gelatin formulation for oral administration to adults of an aqueous extract of roselle of natural origin made up of:
   Aqueous extract of roselle (Hibiscus sabdariffa): 90 ml
   Prebiotic (agave inulin): 40 g
   Encapsulated probiotic mixture: 500 mg of probiotics

| Microorganism | Quantity |
|---|---|
| L. casei | Min. 1.5 × 10\hat{ }10 CFU |
| L. rhamnosus | Min. 5 × 10\hat{ }9 CFU |
| L. lactis | Min. 5 × 10\hat{ }9 CFU |
| L. plantarum | Min. 2.5 × 10\hat{ }10 CFU |
| B. longum | Min. 4 × 10\hat{ }9 CFU |

Protein: range 3.3 g to 20 g

| | |
|---|---|
| L-histidine | 900-5400 mg |
| L-tyrosine | 766.66-4600 mg |
| L-isoleucine | 383.33-2300 mg |
| L-leucine | 766.66-4600 mg |
| L-methionine | 133.33-800 mg |
| L-valine | 383.33-2300 mg |

Caloric value: 200 kcal
Sodium: 70 mg
Potassium: <10 mg
Mixture of micronutrients:

| Micronutrients | Quantity |
|---|---|
| Vitamin B6 | Min. 6.6 mg |
| Vitamin B12 | Min. 6.6 mcg |
| Vitamin B2 | Min. 5.6 mg |
| Vitamin B3 | Min. 66.6 mg |
| Folic acid | Min. 666.66 mg |
| Vitamin A | Min. 3330 mg |
| Vitamin B5 | Min. 23.33 mg |
| Vitamin B1 | Min. 50 mg |
| Vitamin E | Min. 33.33 IU |
| Vitamin K1 | Min. 266.66 mcg |
| Vitamin D3 | Min. 16.66 mg |
| Vitamin C | Min. 200 mg |
| Vitamin H | Min. 333.33 mcg |

This formulation may be presented as a food supplement and to facilitate consumption is presented as gelatin; the formulation above allows adequate absorption of the components in the digestive tract. Agar may also be used as a thickener. The formulation may be presented as a syrup, elixir, suspension or solution.

One of the problems presented by the formulations of the majority of supplements with probiotics is to ensure the bioavailability of said probiotics. In the formulation described, to protect the probiotics in an aerobic medium and guarantee their availability, said probiotics were encapsulated with sodium alginate to form spheres containing the probiotic mixture (L. casei, L. rhamnosus, L. lactis, L. plantarum, B. longum). A study carried out in a digestive system simulator demonstrated the protective effect of the polymer matrix of sodium alginate used in the formulation described on the encapsulated probiotics. Various mixtures of probiotics and various concentrations of the encapsulating solution were evaluated.

The study was carried out using a simulator of the microbiota of patients with an inflammatory process. Sample 1 was the formulation described without the mixture of probiotics being encapsulated; sample 2 corresponds to the formulation described when the mixture of probiotics was encapsulated with a solution of sodium alginate.

Both samples passed through the digestive system simulator and the colony-forming units (CFU) were counted when said CFUs reached the stomach and the small intestine. The table below shows the results.

| | Genus of prebiotic | Quantity in | Section in small |
|---|---|---|---|
| Sample | used | stomach | intestine |
| Sample 1 | Lactobacilllus spp | 22 × 106 | No CFUs |
| Sample 1 | Bifidobacterium ssp | No CFUs | No CFUs |
| Sample 2 | Lactobacilllus spp | 22 × 104 CFU/g | 1 × 104 CFU/g |
| Sample 2 | Bifidobacterium ssp | 4 × 104 CFU/g | 2 × 104 CFU/g |

It can be seen that when the probiotics were not encapsulated, the viability of the microorganisms of the genus Bifidobacterium ssp is zero in the stomach; even when the microorganisms of the genus Lactobacilllus spp reach the stomach in a viable state, when the sample reaches the small intestine their viability is zero. The results for sample 2 demonstrate that both the microorganisms of the genus Bifidobacterium ssp and those of the genus Lactobacilllus spp reached the stomach and the small intestine in a viable state; the count in the small intestine is lower than in the stomach owing to the pH of the stomach which affects the encapsulating polymer matrix.

The alginate solution is a good encapsulant at the concentration used in sample 2. The results in the table above demonstrate that the formulation described allows the probiotics to be in a viable state in the small intestine, and therefore when ingested, said probiotics may be of assistance in re-establishing the intestinal microbiota.

Antioxidants are the largest group of active compounds in foodstuffs. The mechanisms of action of these compounds are to interrupt the propagation of the free radical chain by giving up hydrogen atoms, trapping oxygen molecules and/or chelating activity. These mechanisms are important for health as they control the quantity of free radicals; free radicals are linked to degenerative diseases, including cancer.

Various modifications to these definitions and/or implementations will be quite clear to people skilled in the art, and the generic principles defined herein may be applied to other embodiments without departing from the spirit or scope of the invention. Thus, the present invention is not intended to be limited to the embodiments shown in this document, but the widest possible scope should be granted thereto consistent with the following claims and the principles and novel characteristics described in this document.

Having described my invention sufficiently, I consider the content of the following claims novel and I therefore claim it as my exclusive property:

1. A food supplement formulation to be administered orally in humans with a symbiotic effect to improve the nutritional state and to be used to maintain the intestinal microbiota characterized in that each 100 g comprises:
a. A formulation encapsulated in a polymer matrix of sodium alginate for oral administration to adults of an aqueous extract of roselle of natural origin which contains:
i. Aqueous extract of roselle (Hibiscus sabdariffa): 90 ml
ii. Prebiotic (agave inulin): 40 g
iii. Encapsulated probiotic mixture: 500 mg of probiotics:

| | |
|---|---|
| 1. L. casei | Min. 1.5 × 10 10 CFU |
| 2. L. Rhamnosus | Min. 5 × 10 9 CFU |
| 3. L. lactis | Min. 5 × 10 9 CFU |
| 4. L. plantarum | Min. 2.5 × 10 10 CFU |
| 5. B. longum | Min. 4 × 10 9 CFU |

iv. Protein: range 3.3g -20 g

| | |
|---|---|
| L-histidine | 900-4000 mg |
| L-tyrosine | 766.66-4600 mg |
| L-isoleucine | 383.33-2300 mg |
| L-leucine | 766.66-4600 mg |
| L-methionine | 133.33-800 mg |
| L-valine | 383.33-2300 mg |

v. Caloric value: 200-267 kcal
vi. Sodium: 70 mg
vii. Potassium: <10 mg
viii. Mixture of micronutrients:

| | |
|---|---|
| 1. Vitamin B6 | Min. 6.6 mg |
| 2. Vitamin B12 | Min. 6.6 mcg |
| 3. Vitamin B2 | Min. 5.6 mg |
| 4. Vitamin B3 | Min. 66.6 mg |
| 5. Folic acid | Min. 666.66 mg |
| 6. Vitamin A | Min. 3330 mg |
| 7. Vitamin B5 | Min. 23.33 mg |
| 8. Vitamin B1 | Min. 50 mg |
| 9. Vitamin E | Min. 33.33 IU |
| 10. Vitamin K1 | Min. 266.66 mcg |
| 11. Vitamin D3 | Min. 16.66 mg |
| 12. Vitamin C | Min. 200 mg |
| 13. Vitamin H | Min. 333.33 mcg |

ix. It may also contain up to 8 g of agar as a thickener.
b. The formulation established in letter a may be presented as soft gelatin, syrup, soft gelatin capsules, powder, elixir, suspension, solution or beads.
